(19) United States
(12) Patent Application Publication
(10) Pub. No.: US 2015/0232999 A1
(43) Pub. Date: Aug. 20, 2015

(54) ENVIRONMENTALLY-FRIENDLY INTEGRATED INSTALLATION FOR PRODUCING CHEMICAL AND PETROCHEMICAL PRODUCTS

(71) Applicant: SIEMENS AKTIENGESELLSCHAFT, München (DE)

(72) Inventors: Konrad Busskamp, Rhede (DE); Volkmar Döricht, Munchen (DE); Christoph Kiener, Munchen (DE); Dominik Rohrmus, Munchen (DE); Philipp Emanuel Stelzig, Kammlach (DE)

(21) Appl. No.: 14/426,772

(22) PCT Filed: Aug. 26, 2013
(86) PCT No.: PCT/EP2013/067613

§ 371 Date: Mar. 9, 2015

(30) Foreign Application Priority Data
Sep. 11, 2012 (DE) 102012216090.6

(51) Int. Cl. C25B 1/02; C10G 2/00; C25B 9/00; C01C 1/04; C01B 31/18; B01D 53/02; C10L 3/08; C07C 27/04

(52) U.S. Cl. CPC C25B 1/02; C10L 3/08; C10G 2/30; C10G 2/50; C07C 27/04; C01C 1/04; C01B 31/18; B01D 53/02; C25B 9/00; C01B 2203/068; C01B 2203/061

(57) ABSTRACT

An environmentally-friendly integrated installation includes a combined air separation and carbon dioxide capture installation and an electrolysis unit. In certain embodiments, the integrated installation additionally includes a unit for producing renewable energy. A control unit and a computer program product are provided for the integrated installation. A method for producing chemical products in the integrated installation, and the use of the integrated installation to produce chemical products are disclosed and use as a chemical store for fluctuating renewable energies is disclosed.

The present invention relates to technical possibilities for an environmentally-friendly integrated installation for producing chemical and petrochemical products that are not based on fossil fuels.

### PRIOR ART

For more efficient and more environmentally-friendly utilization of energy from fossil fuels, currently, various techniques are offered, for instance the oxy fuel method, or combustion in an integrated gasification combined cycle (IGCC) installation.

Thus, an IGCC installation, for instance, permits not only an increase in efficiency during the combustion and use of low-grade energy sources, in that the fossil fuel is converted into hydrogen by substoichiometric combustion with oxygen and subsequent introduction into a water gas shift reactor by means of steam, which hydrogen can then be utilized for energy production, but also a method for generating simple chemical products from the reagents used oxygen, water, carbon and nitrogen, in an integrated installation.

In this case, a conventional IGCC installation generally shows at least one air separation unit for providing oxygen and nitrogen, a gasifier for combustion of the fossil fuels, a water gas shift reactor having an installation for removing acid gases such as hydrogen sulfide (acid gas removal unit) for producing hydrogen, and a combined cycle power plant for producing electrical energy from the process heat or the combustion of the products, in particular hydrogen.

In addition, such an IGCC installation can also comprise a unit for binding and storage of carbon dioxide (carbon capture and storage, CCS), wherein carbon dioxide, CO₂, after production of the hydrogen in the water gas shift reactor is removed from the process using an installation for removing acid gases such as hydrogen sulfide (hereinafter also termed water gas shift reactor, if nothing else results from the description) and is stored.

Furthermore, it is also possible, from the synthesis gas SG produced in the water gas shift reactor (WGS), to produce chemical products for use or production of further, possibly complex, chemical products, for example hydrogen (compressed gaseous hydrogen, CGH, or gaseous hydrogen, GH₂), methanol MeOH, fuels T, synthetic natural gas (SNG), ammonia NH₃, or to use the synthesis gas SG itself as a product for further processing.

One example of a conventional IGCC installation is shown in FIG. 1. This comprises an air separation unit ASU (LZA in the figure), a fuel gasification reactor FGR (or gasifier V in the figure), a water gas shift reactor WSR having an installation for removing acid gases such as hydrogen sulfide, and a combined cycle power plant CCPP (GDA in the figure). Oxygen O₂ (gaseous oxygen, GOX) from the air separation unit LZA and optionally steam are fed in such an IGCC installation to the gasifier V in which they are then reacted with a fuel B substoichiometrically to give crude synthesis gas RSG with the main component carbon monoxide CO. The carbon monoxide is reacted with water, for example in the form of steam, in a water gas shift reactor WSR to form synthesis gas SG which contains carbon dioxide and large amounts of hydrogen which then, optionally after separating off carbon dioxide, is used in a combined cycle power plant GDA for energy production. The energy E obtained in this case is then also partly used for operating the air separation unit LZA, since the air separation is an energy-intensive process in which air is separated into oxygen and nitrogen, and also other components. The synthesis gas SG produced in the water gas shift reactor WSR can, however, also optionally be converted in further reactors to form chemical products such as hydrogen H₂, synthetic natural gas or synthetic methane (SNG), methanol MeOH, or fuels T. Using the nitrogen N₂ from the air separation unit LZA, in addition, ammonia NH₃ (water free ammonia WFA or liquid water free ammonia LWA) can also be produced. Also, the use of the synthesis gas SG as product itself is possible. A conventional IGCC installation is known, for example, from WO 2007/094908 A2.

However, a disadvantage with the method in the IGCC installation and further installations which are based on fossil fuels is that carbon dioxide is produced as raw material in the integrated installation by the combustion of fossil fuels and is only available as a raw material subsequently by binding and storage of carbon dioxide, which firstly leads to more complex installations having a plurality of various apparatuses, for example for producing and separation of carbon dioxide, and also to increased environmental pollution by the fossil fuels. Moreover, fossil fuels are burnt, that is to say energy stored in chemical substances is consumed before products of higher value chemically can be produced.

There is therefore a requirement for installations which need not generate intermediates, such as carbon dioxide, for an integrated installation on the basis of fossil fuels. In addition, there is a requirement for integrated installations which are based on more environmentally-friendly raw materials.

This object is achieved by the integrated installation according to claim 1.

An integrated installation of the invention comprises a combined air separation unit and carbon dioxide separation unit 1, and an electrolysis unit 2. In certain embodiments, in addition, a unit 3 for producing renewable energy is provided which, at least in part, provides energy for operating the combined air separation unit and carbon dioxide separation unit 1 and/or the electrolysis unit 2.

An integrated installation according to the invention is distinguished in that it is more environmentally friendly and in certain embodiments can dispense with the use of fossil fuels. In addition, an integrated installation according to the invention permits the complete use of all products produced in the combined air separation unit and carbon dioxide separation unit 1. Furthermore, via the integrated installation according to the invention, in certain embodiments, chemical and petrochemical, CO₂-neutral products can be produced. These products produced can, as “green” products, that is to say as products which are not based on fossil fuels, permit high prices on the targeted markets, for example approximately 800 Euro/t (as of 2012) of green (CO₂-neutral) methanol. In addition, products from the air separation unit can also be obtained as such, such as, for example, oxygen O₂, nitrogen N₂ or argon which can also be marketed as such, wherein for argon, for example, 3000 to 50 000 US Dollars per ton can be achieved, for nitrogen 300 to 500 US Dollars per ton and for oxygen approximately 90 US Dollars per ton (as of 2012). The products produced can then again be converted to electrical energy, without fossil fuels needing first to be used therefor.

In addition, the integrated installation according to the invention permits a flexible passage from electrical energy to chemical energy or products. In addition, the integrated installation according to the invention can be used as a store for fluctuating renewable energy, if, in certain embodiments, the combined air separation unit and carbon dioxide separation unit 1 and/or the electrolysis unit 2 are operated with energy from a unit 3 for producing renewable energy. Therefore, the integrated installation according to the invention differs from a classical store in which that which is to be stored corresponds to that which is withdrawn from the store such as, for example, in petroleum or natural gas tanks or in rechargeable batteries.

The integrated installation according to the invention acts, through operation thereof, overall also as a store, since it can be operated in different ways depending on the supply of energy from the unit 3 for producing renewable energy.

Furthermore, the integrated installation according to the invention can also act as a buffer against the energy fluctuations with the renewable energy produced. This can also be advantageous with regard to the fact that no problems or reduced problems can occur with the grid stabilization in the energy grid, for example in the power grid, such as in the case of direct feed-in of fluctuating energy, for example fluctuating power from renewable energy.

In addition, the present invention comprises a control unit for controlling an integrated installation according to the invention, a method for producing chemical products, using the integrated installation according to the invention, a computer program product for controlling an integrated installation according to the invention, and also the use of the integrated installation according to the invention as a store for fluctuating renewable energies.

### DESCRIPTION OF THE FIGURES

The invention will be described in detail hereinafter with reference to figures, wherein these figures are not intended to limit the scope of the invention.

FIG. 1 shows a schematic structure of a conventional IGCC installation.

FIG. 2 shows a schematic structure of one embodiment of the integrated installation according to the invention.

FIG. 3 shows schematically an exemplary circuit diagram of a control unit according to the invention.

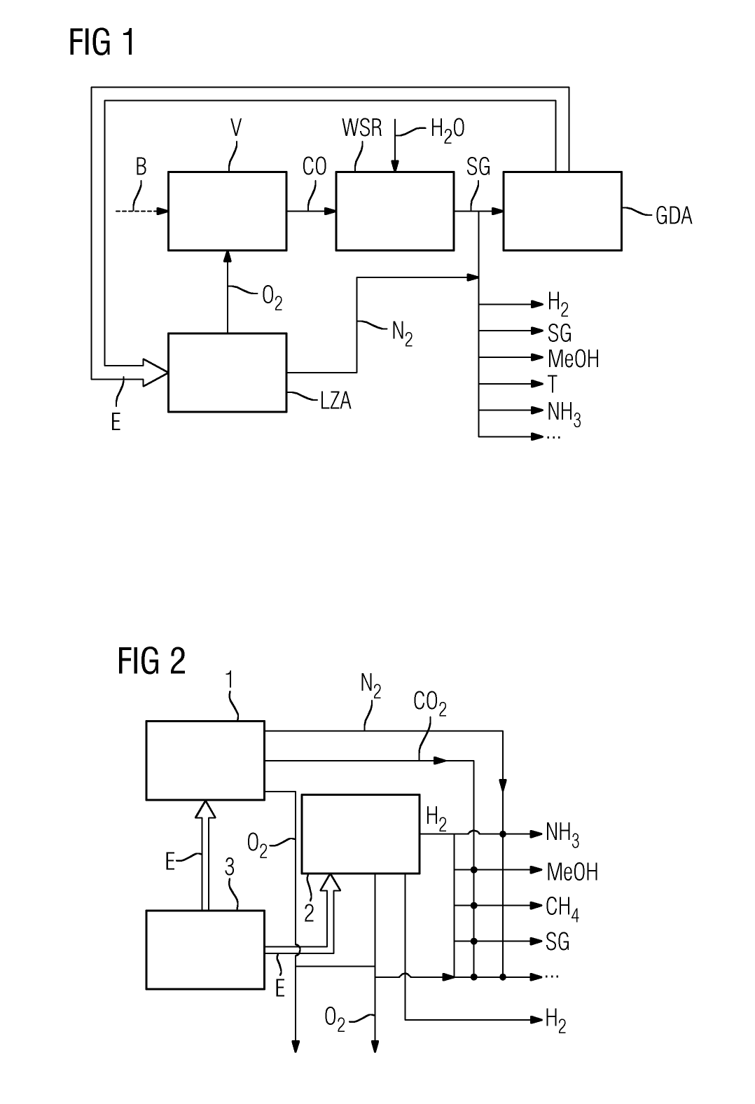

### MORE DETAILED DESCRIPTION OF THE INVENTION

An integrated installation in this case is taken to mean an installation for optionally alternating production of different end products from the same intermediates. That is to say, differing end products can be produced at different time points from the same intermediates, for example carbon dioxide, nitrogen, hydrogen and optionally oxygen. In certain initial forms, in such integrated installations, an integrated process having at least three energy raw materials takes place. In certain embodiments, an integrated installation does not comprise a refinery.

An integrated installation according to the invention has a combined air separation unit and carbon dioxide separation unit 1 and an electrolysis unit 2.

In addition, in certain embodiments in the case of the integrated installation according to the invention, a unit 3 for producing renewable energy can be provided, which unit provides, at least in part, energy for operating the combined air separation unit and carbon dioxide separation unit 1 and/or the electrolysis unit 2. In certain embodiments, the unit 3 for producing renewable energy is integrated into the integrated installation according to the invention or dedicated to the integrated installation. Dedicated in this case means that the energy produced in the unit 3 for producing renewable energy is principally, for example more than 50%, optionally more than 75%, further optionally more than 90%, and preferably completely, apart from losses, for example in transmission, fed into the integrated installation according to the invention. The feed in this case can proceed, for example, via one or more cables. An integration of the unit 3 for producing renewable energy into the integrated installation can proceed, for example, by connection to a further installation section of the integrated installation, for example the combined air separation unit and carbon dioxide separation unit 1 and/or the electrolysis unit 2.

In the case of a connection of the unit 3 for producing renewable energy, in the context of a dedication, in this case, in addition an advantage can result in that only the cable for connecting the unit 3 for producing renewable energy to the further installation sections, for example the combined air separation unit and carbon dioxide separation unit 1 and/or the electrolysis unit 2, transports the fluctuating energy, for example fluctuating power from the renewable energy, without this fluctuating energy being fed into the general energy grid, for example the power grid, or an additional buffer unit being required therefor for trapping large fluctuations in the energy supply. Therefore, in such embodiments, the integrated installation according to the invention can act as a buffer for the fluctuating energy from the unit 3 for producing renewable energy. This can be the case in a similar manner also in the integration of a unit 3 for producing renewable energy into the integrated installation according to the invention, since here also the fluctuations in the production of renewable energy can be trapped by the installation sections and this fluctuating energy is therefore not delivered into the general energy grid. Therefore, here also, no buffer units are required.

The unit 3 for producing renewable energy or regenerative energy is not limited in the context of the invention, and one or more units 3 for producing renewable energy can be integrated into the integrated installation according to the invention or dedicated to the integrated installation. Examples of units 3 for producing renewable energy comprise solar installations and solar cells or photovoltaic installations, solar thermal installations, thermal installations, hydropower plants, run-of-the-river power plants, wind power plants, geothermal installations, installations for producing bioenergy from biomass and tidal power plants. In the context of the invention, it is also conceivable that a plurality of identical or different units 3 for producing renewable energy are used. It is also conceivable that one or more units 3 for producing renewable energy supply the energy only to one unit of the integrated installation according to the invention, for example the combined air separation unit and carbon dioxide separation unit 1 and/or the electrolysis unit 2.

Preferably, the unit 3 for producing renewable energy, under full load, provides at least the energy E which is required for operating the combined air separation unit and carbon dioxide separation unit 3 and/or the electrolysis unit 2 at full capacity of the integrated installation with respect to the production of the products. In particular embodiments, the unit 3 for producing renewable energy, at full load, provides the energy E which is required for operating the combined air separation unit and carbon dioxide separation unit 3 and the electrolysis unit 2 at full capacity of the integrated installation with respect to production of the products. This ensures that in the case of optimal energy production in the unit 3 for producing renewable energy, the combined air separation unit and carbon dioxide separation unit 1 and/or the electrolysis unit 2 do not require further energy from external energy sources, that is energy sources not belonging to the integrated installation according to the invention. In certain embodiments, however, energy from external sources can also be fed to the integrated installation according to the invention, at least in part, for example at less than 75%, preferably less than 50%, and further preferably less than 25%. Preferably, such an energy can be from external energy sources which are not based on fossil fuels, and particularly preferably it can originate from external energy sources which are based on regenerative energy.

Furthermore, the unit 3 for producing renewable energy can also supply energy E to other installation sections of the integrated installation according to the invention, for example for use in pumps and/or compressors, or for use in reactors in product production, or for operating electrical heaters in the process, or trace heating for heating pipelines, for producing light and heat in buildings, for operating other electrochemical reactions.

The combined air separation unit and carbon dioxide separation unit 1 can be used for providing carbon dioxide CO₂ and/or for providing nitrogen N₂ (gaseous nitrogen, GAN) for the synthesis of chemical products, for example also ammonia NH₃. In this case, the type of the combined air separation unit and carbon dioxide separation unit 1 is in no way restricted as long as air separation and separation of carbon dioxide are permitted in the installation. Combined air separation and carbon dioxide separation units, however, generally require a large amount of energy in order to separate nitrogen, carbon dioxide and oxygen, and also the further components of the air by compression and subsequent cooling, in order that these components of the integrated installation can be available. For this reason, in certain embodiments, it can be advantageous to provide a part of the energy required or all of the energy required of the combined air separation unit and carbon dioxide separation unit 1 by the unit 3 for producing renewable energy, in order thereby to decrease or minimize and optionally reduce to zero the demand for this energy from other sources.

The carbon dioxide separation in the combined air separation unit and carbon dioxide separation unit 1 is not particularly restricted and can proceed, for example, in a usual manner via adsorption and stripping in the combined air separation unit and carbon dioxide separation unit 1. Also, the separation of the carbon dioxide can proceed, for example, via carbon dioxide absorbers from which the carbon dioxide can then be desorbed again later. Examples of methods and devices for carbon dioxide separation are also known from carbon dioxide removal in IGCC installations. For example, carbon dioxide can be removed, for instance, by a Rectisol method using methanol at about −40° C., a Selexol method using polyglycol ethers at approximately 4° C., scrubbing with a potassium carbonate solution, a pressurized water scrubbing, or a lithium scrubbing, reversible binding to carbon dioxide-storing substances such as lithium silicate, etc.

The carbon dioxide can be obtained in the combined air separation unit and carbon dioxide separation unit 1 from the ambient air or from exhaust gases of combustion products or other carbon dioxide-containing gas mixtures. Ambient air in this case is the air in the direct surroundings of the combined air separation unit and carbon dioxide separation unit 1, for example the air in the atmosphere at the site of the integrated installation according to the invention.

In certain embodiments, air or an exhaust gas having a carbon dioxide concentration of greater than or equal to the concentration of the carbon dioxide in the atmosphere, preferably greater than or equal to 390 ppm and having a carbon dioxide concentration of not greater than 15% by volume, preferably less than 12% by volume, for example less than 11% by volume or less than 10% by volume, and preferably ambient air, can be fed to the combined air separation unit and carbon dioxide separation unit (1).

Since the carbon dioxide from the combined air separation unit and carbon dioxide separation unit 1 is used for the production of chemical products, in certain embodiments, it is preferred that air having a higher proportion of carbon dioxide is introduced into the combined air separation unit and carbon dioxide separation unit 1, for example also from combustion processes of fossil fuels in power plants or from chemical processes in which carbon dioxide is formed as a byproduct. A higher proportion in this case is, for example, more than the carbon dioxide proportion in the air of the atmosphere, 390 ppm volume fraction (as of 2011), optionally more than 500 ppm, further optionally more than 700 ppm volume fraction, for example more than 0.1% by volume or more than 1% by volume.

The oxygen produced in the combined air separation unit and carbon dioxide separation unit 1 can, in certain embodiments, be used for producing energy, for example in combustion and/or oxidation processes, and/or for producing chemical products in the integrated installation according to the invention. In certain embodiments, it can also be stored in liquid oxygen stores. Likewise, the oxygen can be marketed as such as a product. In addition, the products that are further produced in the combined air separation unit and carbon dioxide separation unit 1, such as, for example, nitrogen and argon, which can be obtained in an air separation, can be obtained as such and optionally stored.

The air separation can proceed in certain embodiments in the combined air separation unit and carbon dioxide separation unit 1, for example by the Linde method, wherein after removal of oxygen and nitrogen, approximately 4% by volume of carbon dioxide remain in the air, and optionally after a further separation of argon, approximately 95% by volume or approximately 98% by weight carbon dioxide remain in the air, which then in certain embodiments can be separated in the combined air separation unit and carbon dioxide separation unit 1. However, it is also possible already to separate off the carbon dioxide before a separation of argon or nitrogen and/or oxygen or at another time point. An argon separation can, in certain embodiments, such as in a Linde method, be obtained, for example, in a separate argon column, in addition to the main rectification column, and the argon can then also be further purified.

Furthermore, the integrated installation according to the invention has an electrolysis unit 2 in which, for example from water, hydrogen H₂ and oxygen O₂ are produced. This electrolysis unit 2, in various embodiments, can also be operated with the unit 3 for producing renewable energy.

Also, the oxygen produced in the electrolysis unit 2 can, in certain embodiments, be used for producing energy, for example in combustion and/or oxidation processes, and/or for producing chemical products in the integrated installation according to the invention. In certain embodiments, it can also be stored in oxygen stores, for example after compression as liquid oxygen.

It is conceivable, for example in certain embodiments, that the oxygen from the combined air separation unit and carbon dioxide separation unit 1 and/or the electrolysis unit 2 is used for producing chemical products, for example in an oxidation, or a combustion, for example of fossil fuels or of hydrogen, for energy generation, for example in an IGCC installation, or coal power plants.

The hydrogen, H₂, produced in the electrolysis unit 2 can be provided for producing hydrocarbons or further products from the carbon dioxide produced in the combined air separation unit and carbon dioxide separation unit 1, or can be obtained as product as CO₂-neutral hydrogen.

In certain preferred embodiments, the electrolysis unit 2 can be started very rapidly, i.e., started up, for example within a period of up to 50 s, but also in the range of several minutes, for example 5 or 10 minutes, or 15 minutes, up to half an hour in certain cases, in order to be able to react rapidly to fluctuations in the energy supply from the unit 3 for producing renewable energy, as is possible, for example, using an electrolysis with proton-exchange membrane (PEM, or polymer electrolyte membrane), or the conventional alkaline atmospheric electrolysis or pressure electrolysis. For instance, such energy fluctuations occur, for example in the use of wind energy, in the unit 3 for producing renewable energy, as is conceivable, for instance, in the case of storms, but also sometimes first in the range of several minutes. In certain embodiments, however, electrolyses having a slower starting time such as, for example, in certain types of solid oxide electrolysis, are sufficient, for instance in the range of some minutes up to several hours. Such slower electrolysis units achieve in this case an increase in total output which can be taken up by the unit, but are not so suitable for trapping rapid fluctuations in the energy, and so in this case, the fluctuations from the unit 3 for producing renewable energy can firstly be trapped by the combined air separation unit and carbon dioxide separation unit 1.

In a rapid electrolysis which is controllable in the millisecond range, then, for example, the uptake of electrical energy from renewable sources and the associated production of oxygen and hydrogen in the electrolysis unit 2 can also be controlled in the millisecond range, which permits control of the input, for example into the product production.

In addition, the integrated installation can contain various stores for storing reagents, for example oxygen and/or hydrogen and/or nitrogen and/or carbon dioxide, and also further substances, such as, for example, cooling water, and also products, for example synthesis gas SG, carbon monoxide, carbon dioxide, synthetic natural gas, synthetic methane from methanization, various hydrocarbons, for example alkanes according to the Fischer-Tropsch synthesis or alkenes or alkynes, for example methane, ethane, propane, butane, aldehydes, for example from the oxo synthesis, ketones, carboxylic acids such as formic acid and acetic acid, ammonia, amine compounds, nitric acid, fuels T, alcohols such as, for example, methanol MeOH, ethanol, or else oxygen, nitrogen and/or argon, etc. from which in the combined air separation unit and carbon dioxide separation unit 1, and also optionally raw materials for the unit 3 for producing renewable energy, such as, for example, in biomass power plants. With the aid of these stores, it can then be possible to shut off individual parts of the integrated installation according to the invention without the integrated installation itself needing to be shut down. For instance, it is possible, for example, in the event of an excess of renewable energy from the unit 3 for producing renewable energy, to use this excess for producing hydrogen and oxygen in the electrolysis unit 2 in such a manner that then hydrogen and oxygen can be stored. Stored hydrogen can then also be provided for producing, for example, synthesis gas SG, if at a low energy from the unit 3 for producing renewable energy, less hydrogen is produced in the electrolysis unit 2. The type of the stores is not restricted, and usual stores, for example tanks for gases and/or liquids or else stores for solids, can be present. Also, a plurality of stores can be present for one reagent or one product, for example oxygen, nitrogen, hydrogen, carbon dioxide etc. Likewise, the stores, in particular gas stores, can have valves for storage of reagents and/or products.

The stores can therefore also serve for trapping the dynamic, for example owing to the fluctuating energy from the unit 3 for producing renewable energy, or the various stocks of reagents or the demand for products. For example, through an oxygen store, the supply with oxygen can be ensured when the renewable energy from the unit 3 for producing renewable energy is insufficient for operating the combined air separation unit and carbon dioxide separation unit 1 and/or the electrolysis unit 2, in such a manner that energy can then be used from a combustion or oxidation of fuels, for example also stored hydrogen or other fuels, with the oxygen for operating for example the combined air separation unit and carbon dioxide separation unit 1.

The various stores can be connected via various tubes, which are not restricted, for the transport of reagents and/or products to the various installation sections of the integration installation according to the invention, for example the combined air separation unit and carbon dioxide separation unit 1 and/or the electrolysis unit 2, which can also have various valves.

Likewise, the various installation sections of the integrated installation according to the invention are connected, where required, via various tubes, as is the case also in conventional integrated installations, for example also various reactors for product production to one another and/or to the combined air separation unit and carbon dioxide separation unit 1 and/or the electrolysis unit 2. It is also possible that in certain embodiments of the integrated installation according to the invention, in the unit 3 for producing renewable energy materials are produced, for example methane in biogas installations, which are introduced as reagents and/or products into the integrated installation according to the invention, for example into one or more stores, or else into other installation sections, for example when hydrogen is produced, into product production, via tubes.

In addition, an integrated installation according to the invention can additionally comprise all further components or installation sections which are used in conventional integrated installations, for example gas purification systems/gas purification units and/or heat recovery systems/heat recovery units. For example, impurities can be removed from the air by corresponding purification systems/purification units upstream and/or downstream of the combined air separation unit and carbon dioxide separation unit 1. The heat recovered in the heat recovery systems can be used, for example, in the reactors for producing the products.

The integrated installation according to the invention can require a relatively complex control of the installation in certain embodiments, for example also in connection with fluctuating energy supply from a unit 3 for producing renewable energy, but also the carbon dioxide supply from the combined air separation unit and carbon dioxide separation unit 1. Such a control of the integrated installation according to the invention can proceed, for example, via a control unit according to the invention. In particular, a control unit according to the invention controls the energy supply and/or the reagent streams and/or the product streams and/or optionally the reagent storage and/or the product storage within the integrated installation according to the invention. The control can, in certain embodiments in the control logic SL, be adjusted to the market demand for certain products, in that, for example, market models MM are integrated in the control software SSO and these are matched with the strategy ST of a trader/dealer TR, in order to control the installation in accordance with demand also. In addition, the control software SSO can have projections PR for the installation, which then likewise can have an influence on the installation control.

Such projections PR can also comprise projections on the availability of energy from the unit 3 for producing renewable energy, for example on the basis of wind data in the case of wind power plants, or on the basis of sunshine intensities in the case of solar power plants. In addition to the control software SSO, the controller of the integrated installation according to the invention can also have a control appliance which can be provided as a balancing controller AR, and ensures that the installation is operated within the component-specific and rule-specific limits in order to prevent defects and/or breakdowns in individual installation sections. Furthermore, the controller according to the invention, in certain embodiments, also has a control system SSY for the individual installation sections and their connections, valves, etc., which controller monitors and administrates the installation configuration AK, that is to say the units thereof. The control software SSO in this case can then send targets ZV to the control system SSY which then controls individual units of the integrated installation correspondingly via the unit controller ES, and the control system SSY can in addition communicate the current states IS of the respective units to the control software. In the control software SSO, then the individual units can then be compared also with respect to external circumstances, such as the availability of energy from the unit 3 for producing renewable energy, or the store states in individual reagent and/or product stores, which can have limiting effects, in such a manner that then adaptation of the controller to these circumstances can proceed.

In certain embodiments, the control unit according to the invention can control the energy supply of individual installation sections of the integrated installation, for example the combined air separation unit and carbon dioxide separation unit 1 and/or the electrolysis unit 2 and/or optionally also the unit 3 for producing renewable energy, for example for starting the unit 3 for producing renewable energy in cases in which it is necessary, and/or the reagent streams and/or the product streams to and/or from individual installation sections, for example the combined air separation unit and carbon dioxide separation unit 1 and/or the electrolysis unit 2 and/or the reactors for producing products and/or optionally also the unit 3 for producing renewable energy.

Using the integrated installation according to the invention, chemical products can be produced as stores for renewable energy. For example, carbon dioxide and nitrogen and optionally oxygen can be produced as raw materials/intermediates in the combined air separation unit and carbon dioxide separation unit 1 and hydrogen and optionally oxygen as raw materials/intermediates in the electrolysis unit 2 and converted to chemical products. In the method according to the invention, it is also possible that additionally carbon dioxide from external sources, for example from other combustion processes, or from external stores or from further chemical processes which produce carbon dioxide, is reacted with the raw materials/intermediates produced in the combined air separation unit and carbon dioxide separation unit 1 and/or the electrolysis unit 2 to form chemical products.

As reagents in the production of chemical products, also the products proceeding from the reaction of the raw materials/intermediates, which products can be reacted further, can be considered, such as, for example, carbon monoxide, ammonia or methane, which can be reacted further. Also, reagents from, for example, the unit 3 for producing renewable energy such as, for example, gas products from a biogas installation are conceivable.

In the method according to the invention, the raw materials/intermediates are converted in the integrated installation according to the invention into products, for example synthesis gas SG, carbon monoxide, carbon dioxide, synthetic natural gas, synthetic methane, various hydrocarbons, for example alkanes according to the Fischer-Tropsch synthesis, or alkenes or alkynes, in particular methane, ethane, propane, butane, aldehydes, for example from the oxo synthesis, ketones, carboxylic acids such as formic acid and acetic acid, ammonia, amine compounds, nitric acid, fuels T, alcohols such as, for example, methanol MeOH, ethanol etc. In certain embodiments, the products can be obtained using the energy from the unit 3 for producing renewable energy. The energy from the unit 3 for producing renewable energy can in this case be used, for example, for the combined air separation unit and carbon dioxide separation unit 1 and/or the electrolysis unit 2, depending on availability of the renewable energy, which can also be, for example, fluctuating, but, in certain embodiments, can also be available constantly. In certain embodiments, the energy from the unit 3 for producing renewable energy, however, can be fluctuating, in such a manner that the integrated installation according to the invention acts as a buffer for this fluctuating energy.

Owing to the use of renewable energy from the unit 3 for producing renewable energy in the production of products in the integrated installation according to the invention, products are obtained which are based, preferably completely, on this renewable energy, and therefore are considered to be “green”, i.e. CO₂-neutral, products or energy, which is accompanied by an added value for the products and is also more environmentally friendly. For example, for green (CO₂-neutral) methanol MeOH, prices up to 800 Euros/t can be obtained, that is to say an added value in the product. Likewise, when the renewable energy from the unit 3 for producing renewable energy is used in the combined air separation unit and carbon dioxide separation unit 1 and/or the electrolysis unit 2, CO₂-neutral oxygen can be obtained. Also, in addition, from the electrolysis unit 2, when renewable energy from the unit 3 for producing renewable energy is used, CO₂-neutral “green” hydrogen can be obtained which can be obtained as such as a product, or can be used in the production of other “green” chemical products in the integrated installation according to the invention, such as alkanes or synthetic natural gas, etc.

This therefore represents one route for converting renewable energy into valuable chemical products, that is to say what is termed a switching point between principally renewable electricity and chemical processes.

In contrast thereto, for example hydrogen in conventional IGCC installations is not CO₂-neutral, since the energy for production thereof from water in the water gas shift reactor originates from the conventional IGCC installation. Also, the electrical energy for operating an air separation unit in conventional IGCC installations is obtained within the IGCC installation, in such a manner that the air separation unit in a conventional IGCC installation cannot be considered as CO₂-neutral.

In the integrated installation according to the invention, therefore, the use of renewable energies, for example also in a large amount of more than 50%, preferably more than 75%, further preferably more than 90%, and in particular, in certain embodiments, completely, can take place, and also the production of potentially CO₂-neutral products, preferably CO₂-neutral products. This also results in novel application possibilities and business fields for the operators of integrated installations.

In addition to the buffer action of the integrated installation according to the invention with respect to fluctuating energy from the unit 3 for producing renewable energy, a buffer action with respect to reagents and/or products can also be achieved in the integrated installation according to the invention, in that, for example, depending on energy input, various installation sections of the integrated installation, such as, for example, the combined air separation unit and carbon dioxide separation unit 1 and/or the electrolysis unit 2, are reduced or not operated at all for a certain time, for example up to the renewed availability of more renewable energy or until the need for a reagent and/or product from one or more stores.

In addition, the storage action in the integrated installation according to the invention can also be considered to be that a raw material, such as carbon dioxide, is converted at least in part by renewable energy into a higher-value substance or a higher-value product having a higher calorific value, for example methane or methanol MeOH. These higher-value products can then be used for energy production, again with energy production in comparison to the original raw material, in such a manner that they can therefore be considered as higher-grade energy stores for the renewable energy used in the integrated installation according to the invention.

Nevertheless, in the integrated installation according to the invention, energy from external sources, for example a conventional combustion/gasification in power plants or nuclear power plants or other sources, can be used for operating the integrated installation when no renewable energy can be produced, for example in the case of still air in the use of wind power plants, or at night or in the case of a covered sky when solar power plants are used.

An integrated installation according to the invention can be operated in different phases, depending, for example, on the energy feed from the unit 3 for producing renewable energy and/or the presence of stores for storing reagents and/or products:
When little renewable energy from the unit 3 for producing renewable energy is present, the energy consumption can be decreased, for example, by throttling the production of chemical products and/or decreasing the load on the combined air separation unit and carbon dioxide separation unit 1 and/or the electrolysis unit 2 and/or optionally using stored reagents from stores.
When renewable energy is available in a usual extent, it can be used for operating the combined air separation unit and carbon dioxide separation unit 1 and/or for operating the electrolysis unit 2. Also, the production of chemical products can proceed here in a usual extent.
In the event of a surplus of renewable energy, in addition to the usual operation, for example a surplus of oxygen and/or hydrogen can be produced which can then be stored.
In the case of an extreme lack of renewable energy, in certain embodiments, energy from external sources can also be used for operating the combined air separation unit and carbon dioxide separation unit 1 and/or the electrolysis unit 2.

Depending on the stocks of reagents or products in stores, an integrated installation according to the invention can also be operated in other ways, and also adapted to the desires of clients, for example consumers of the chemical products that are produced.

Also with reference to these different phases, it can be recognized that the integrated installation according to the invention can become more complex, which requires a more complex controller with an improved control unit which also automatically ensures the installation control in certain embodiments. For the control, therefore, in certain embodiments, various sensors, for example for measuring the available renewable energy from the unit 3 for producing renewable energy and/or also the efficiencies in the various installation sections and/or the statuses of the stocks in the various stores and/or the mass transport streams are also required, which also can be determined by the control unit according to the invention and therefore also lead to an automatic adaptation of the integrated installation by the control unit on the basis of the data from the sensors. This can also proceed via the computer program product according to the invention for controlling the integrated installation.

With the integrated installation according to the invention, it is, in addition, also possible to react to further fluctuations, for example in the production of product, for instance of ammonia. For example it is also possible, in the absence of hydrogen, still to pass nitrogen from the combined air separation unit and carbon dioxide separation unit 1 into an installation for ammonia synthesis, since nitrogen does not damage a catalyst used for the synthesis of ammonia in such an installation, or to keep the catalytic reactor of the ammonia synthesis operationally ready without substantial cooling down without addition of hydrogen/nitrogen and without takeoff of ammonia.

In addition, the present invention comprises a computer program product, optionally a non-transitory computer program product, which is used in the control unit according to the invention for controlling the integrated installation according to the invention.

For example, the computer program product can comprise the control software SSO of the controller according to the invention. In this case, the controller, in certain embodiments, can be directed towards the market demand for certain products, in that, for example, market models MM are integrated in the control software SSO and these are compared with the strategy ST of a dealer TR by the computer program product, in order to control the installation according to demand also. In addition, the control software SSO can have projections PR for the installation which then likewise can act on the installation controller. Such projections PR can also comprise projections on the availability of energy from the unit 3 for producing renewable energy, for example on the basis of wind data in the case of wind power plants. In addition, the control software SSO can provide targets ZV for the entire installation or individual installation sections, and compare these with current states IS from the control system SSY and thereby coordinate the controller in the system.

In the control software SSO, in addition, comparison of the individual units, also with respect to external circumstances such as the availability of energy from the unit 3 for producing renewable energy or the store states in individual reagent and/or product stores can proceed, which can act in a limiting manner, and so then an adaptation of the controller to these circumstances can proceed.

In certain embodiments, the control software SSO can also communicate with a control appliance which can be provided as a balancing controller AR and ensures that the installation is operated within the component- and rule-specific limits, in order to avoid defects and/or breakdowns in individual installation sections. Such a balancing controller AR, in certain embodiments, can also be integrated into a computer program product according to the invention.

Hereinafter, the invention will be described on the basis of an exemplary integrated installation, as is shown in FIG. 2 but which in no way restricts the invention.

In FIG. 2, an exemplary embodiment of an integrated installation according to the invention is shown, which, in addition to a combined air separation unit and carbon dioxide separation unit 1, also has a unit 3 for producing renewable energy. The unit 3 for producing renewable energy provides energy E, for example for the combined air separation unit and carbon dioxide separation unit 1 and the electrolysis unit 2, in such a manner that the carbon dioxide introduced into the product production is CO₂-neutral. Likewise, the nitrogen N₂ from the combined air separation unit and carbon dioxide separation unit 1, which can be used, for example, for later ammonia production, is CO₂-neutral. Also, the hydrogen from the electrolysis unit 2 is CO₂-neutral in this example. The raw materials/intermediates carbon dioxide, nitrogen and hydrogen can then be used for producing chemical products in, for example, separate devices of the integrated installation, for example reactors, which are shown in FIG. 2 as points on the crossings of the material streams, or, for example, the hydrogen H₂ can also be obtained as a product. Also, oxygen O₂ can be obtained as a product from the combined air separation unit and carbon dioxide separation unit 1 and/or from the electrolysis unit 2, or used for producing further products. Exemplary chemical products can be methanol MeOH, synthesis gas, methane, CH₄, or fuels T, etc. Also, ammonia NH₃ can be obtained as a product.

In certain embodiments of such an installation, however, the energy supply for the combined air separation unit and carbon dioxide separation unit 1 and/or the electrolysis unit 2 can be provided in part or in whole from external energy sources.

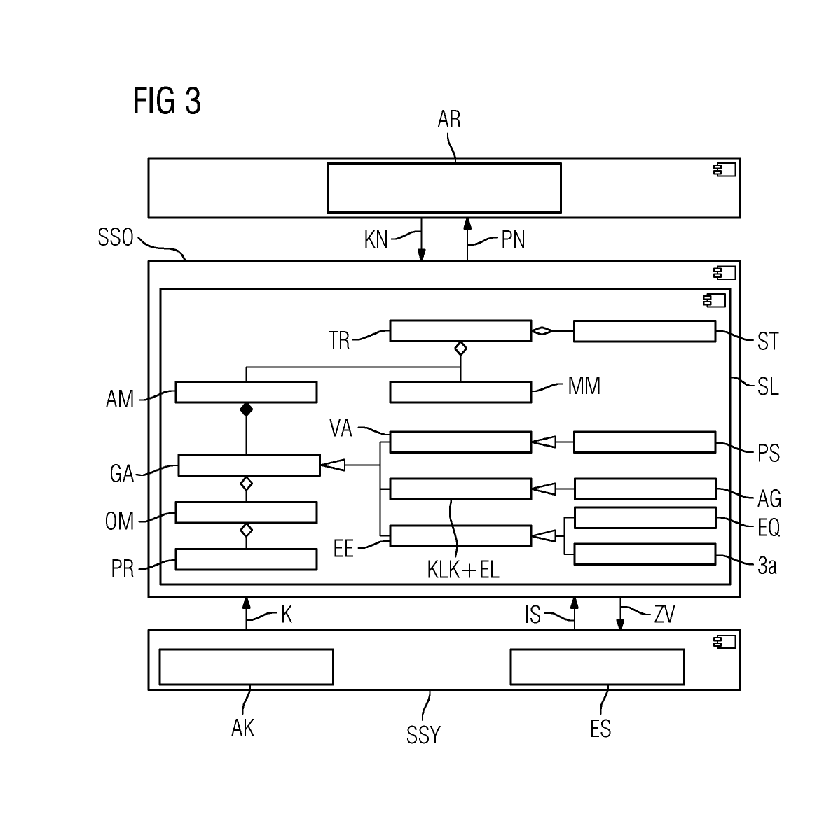

FIG. 3 shows, furthermore, an exemplary schematic of a controller according to the invention.

In the controller of FIG. 3, the entire installation is controlled via control software SSO which is determined not only by the strategy ST of the dealers TR with respect to the product demand, but also the installation model AM and the market model MM for the product market. The installation model AM comprises the entire installation GA in abstract form and is based not only on projections PR for the entire installation which act on the installation via an operation manager OM, but also on the individual installation sections, such as, for example, the production of chemical products in the integrated installation VA and also the product scenarios PS therefor, the combined air separation unit and carbon dioxide separation unit KLK (abstract in the control software SSO) and the electrolysis unit EL (abstract in the control software SSO), for example the respective load degree AG, and the producers of electrical energy EE for the installation operation, such as, for example, via external sources EG or the unit 3 for producing renewable energy in the control software SSO (abstract as 3a).

The control software SSO then sends targets ZV to the control system SSY which then controls the respective unit controllers ES correspondingly, and also reports the current state IS to the control software SSO. In addition, the control system SSY also reports the installation configuration AK as configuration K.

Furthermore, the controller of FIG. 3 further has a balancing controller AR which controls the installation within preset limits via, for example, component-specific and/or rule-specific rules, in order that fault-free installation operation can be ensured. In this case, the balancing controller AR transmits coordination news KN to the control software SSO for maintaining these limits, and receives protocol news PN, which then serve for monitoring the limits and can form the basis for further coordination.

Using the integrated installation according to the invention, CO₂-neutral “green” products can be obtained. In addition, the integrated installation according to the invention can serve as buffer with respect to fluctuating energy supply from a unit for producing renewable energy. Also, the integrated installation according to the invention can serve as buffer for reagents and/or chemical products in the integrated installation.

1. An integrated installation for producing chemical products, the installation comprising:
a combined air separation unit and carbon dioxide separation unit configured for obtaining at least carbon dioxide and nitrogen; and
an electrolysis unit for obtaining hydrogen;
wherein at least the carbon dioxide obtained in the combined air separation unit and carbon dioxide separation unit is used for producing the chemical products.

2. The integrated installation as claimed in claim 1, further comprising a unit for producing renewable energy configured and connected for providing energy to and for operating at least one of the combined air separation unit and carbon dioxide separation unit and the electrolysis unit.

3. The integrated installation as claimed in claim 1, wherein the combined air separation unit and carbon dioxide separation unit are configured to receive air or an exhaust gas having a carbon dioxide concentration of greater than or equal to the concentration of the carbon dioxide in the atmosphere and are fed to the combined air separation unit and carbon dioxide separation unit.

4. The integrated installation as claimed in claim 1, further comprising stores for storing reagents and/or products produced by the integrated installation.

5. The integrated installation as claimed in claim 4, further comprising the integrated installation has a control unit configured to optimize and control the energy supply and/or the reagent streams and/or the product streams and/or the reagent storage and/or the product storage.

6.-8. (canceled)

9. A method for producing chemical products, comprising producing intermediates for the production of the chemical products, at least in part, in the combined air separation unit and carbon dioxide separation unit and the electrolysis unit of the integrated installation as claimed in claim 1 and converting the intermediates to the chemical products in the integrated installation.

10. The method as claimed in claim 9, wherein carbon dioxide and nitrogen from the combined air separation unit and carbon dioxide separation unit and hydrogen from the electrolysis unit are converted to the chemical products.

11. The method as claimed in claim 10, further comprising reacting carbon dioxide from external sources with the intermediates obtained in the combined air separation unit and carbon dioxide separation unit and the electrolysis unit to form the chemical products.

12. The method as claimed in claim 9, further comprising using oxygen from at least one of the combined air separation unit and carbon dioxide separation unit and from the electrolysis unit for at least one process of producing energy and producing the chemical products.

13. The method as claimed in claim 12, further comprising using the energy produced by the use of oxygen, at least in part, for operating at least one of the combined air separation unit and carbon dioxide separation unit and/or the electrolysis unit of the integrated installation.

14.-16. (canceled)

17. The integrated installation as claimed in claim 3, wherein the carbon dioxide concentration is greater than or equal to 390 ppm and having a carbon dioxide concentration of not greater than 15% by volume.

18. The integrated installation as claimed in claim 3, wherein ambient air is fed to the combined air separation unit and carbon dioxide separation unit.

19. A non-transitory computer program product for controlling an integrated installation and stored on a non-transitory storage medium, the program comprising computer readable instruction which when executed in a control unit of an integrated installation for producing chemical products, causes the installation to perform the method as claimed in claim 9.
